(19) United States
(12) Patent Application Publication
(10) Pub. No.: US 2026/0256594 A1
(43) Pub. Date: Sep. 3, 2026

(54) Universal Humeral Stem System

(71) Applicant: Convertatek Shoulder Prosthesis Solutions, LLC, Old Tappan, NJ (US)

(72) Inventors: Raphael S.F. Longobardi, Old Tappan, NJ (US); Roy C. Wiley, Warsaw, IN (US)

(21) Appl. No.: 19/163,029

(22) PCT Filed: Mar. 8, 2024
(86) PCT No.: PCT/US2024/019189

§ 371 Date: Sep. 8, 2025

Related U.S. Application Data
(60) Provisional application No. 63/450,865, filed on Mar. 8, 2023.

(51) Int. Cl. A61F 2/40; A61F 2/30

(52) U.S. Cl. CPC A61F 2/4059; A61F 2002/30011; A61F 2002/3006; A61F 2002/30604; A61F 2002/4062

(57) ABSTRACT

A universal humeral stem (100) includes an anchoring functionality for use in a universal shoulder prosthesis system that allows for modular humeral components to be exchanged while leaving the humeral stem in place during a transition from traditional anatomic total shoulder replacement to a reverse total shoulder replacement. The humeral stem (100) includes a proximal body (120) with a mounting base (122), a cloverleaf neck (130), and a shaft (140) and an optional distal stem (150) attached to the proximal body (120). In a further embodiment, the proximal body (120A) and the distal stem (150A) are separate components allowing for matching different distal stems based on length and diameter for a particular patient anatomy.

This application claims the benefit of U.S. provisional patent application number 63/450,865 entitled “Universal Humeral Stem System”, which was filed on Mar. 8, 2023, and is hereby incorporated by reference.

### I. FIELD OF THE INVENTION

The invention relates to a universal humeral stem, for example of the type that could be used in a modular shoulder prosthesis system that provides for flexibility in shoulder replacements and allows for a more efficient switch for a patient between a traditional anatomic Total Shoulder Replacement (ta-TSR) to a reverse Total Shoulder Replacement (r-TSR). In a further embodiment, at least one modularity aspect of the universal humeral stem allows for its use in fractures and revision settings.

### II. BACKGROUND OF THE INVENTION

TSRs have evolved over the last 70 years, with the greatest degree of its evolution occurring within the past 20 years. The understanding of the complexity of the shoulder has resulted in the ability to better treat the multiple conditions that afflict the shoulder. Glenohumeral arthritis ranges from simple to complex due to etiology and deformity. Post traumatic glenohumeral arthritis, along with the deformity of both the glenoid and humeral head present challenges for the shoulder arthroplasty surgeon. Similarly, the problem of rotator cuff deficiency and rotator cuff arthropathy has resulted in the development of treatment and prosthetic designs specific to address the loss of the main motors of the shoulder.

Currently, there are two types of TSR-traditional anatomic total shoulder replacement (ta-TSR) and reverse total shoulder replacement (r-TSR). Ta-TSR resurfaces of the humeral head and glenoid in the setting of an intact and functioning rotator cuff. Glenohumeral arthritis has been treated with ta-TSR, the current gold standard being the resurfacing of the humeral head with a stemmed or metaphyseal component along with a replacement of the humeral head articular portion with a Cobalt-Chromium (Co—Cr) implant. The humeral components allow for appropriate sizing of the head in diameter and thickness to match the resected articular surface of the patient.

The most common cause of failure of the ta-TSR is due to glenoid loosening secondary to rotator cuff failure/tear. The resulting superior migration of the humeral head, with concomitant change in the center of rotation (C.O.R.) from rotator cuff failure produces edge loading of the glenoid component. This asymmetric mechanical loading results in rocking and loosening of the polyethylene prosthesis from the cement and bone of the glenoid.

R-TSR evolved from the specific abnormal mechanics of the rotator cuff deficient shoulder, as previously described. In the rotator deficient condition, the deltoid muscle becomes the predominant motor, but in an inefficient manner. The deltoid muscle contraction functions to result in “hinged abduction” of the humeral head/humeral shaft. The humeral head and greater tuberosity lever on the undersurface of the acromion and superior portion of the glenoid. Ta-TSR is contra-indicated in the setting of rotator cuff deficiency, due to the known catastrophic results to the glenoid component.

The development of the r-TSR addresses the rotator cuff deficient, painful arthritic shoulder. The design of r-TSR is to maximize deltoid fiber length to allow more efficient contraction and function of the deltoid in elevation of the arm. The prosthetic components are designed to change the C.O.R. to one that is more inferior and medial to the native joint.

The design of r-TSR has also evolved over the past 20 years. The original “Grammont” style sought to inferiorly displace the humerus to maximize deltoid fiber length; this resulted in inferior scapular notching, leading to failure. The current revised designs include a C.O.R. which is more lateral and inferior to the native C.O.R. The implant design is for an in-growth trabecular metal baseplate with locking screws to secure the component to the bony glenoid. The relatively minimally curved glenoid is replaced with a glenosphere: a solid Co—Cr semi-spherical to ¾ spherical surface that attaches to the base-plate. This is typically through a combination of a Morse taper fit and center screw fixation. The glenosphere is typically inserted at an inferiorly directed version angle, between 5-10 degrees. This allows for inferior offset of the humerus, elongation of the deltoid muscle fibers and a joint reactive force in line with prosthetic alignment.

R-TSR already have asymmetric, higher shear and higher loading of the glenoid component, called the glenosphere and baseplate construct. Despite these greater loads, in growth metal baseplates with locking screws are not the cause of failure, due to the excellent bone incorporation and stability.

The r-TSR has a different humeral component design as well. Where the ta-TSR has the Co—Cr humeral head, the r-TSR had the Co—Cr glenosphere attached to the glenoid. The humerus had a stemmed component but attached to the top is a polyethylene cup or humeral cup to articulate with the glenosphere. The components, specifically glenosphere sizing and humeral cup sizing, allow for multiple permutations to achieve the most successful and stable construct.

### III. SUMMARY OF THE INVENTION

In at least one embodiment, a humeral stem includes a proximal body having a mounting base with a substantially planar surface with optional notches extending down from a peripheral edge of the mounting base and an optional receiving cavity configured to receive a modular humeral component, a neck extending from the mounting base, the neck having three cloverleafs evenly spaced on three sides or four cloverleafs evenly spaced around the neck such that a cross-section resembles a circle with arcuate areas around the circle's perimeter, at least three of the cloverleafs (or optionally one or two of the cloverleafs) include one or more passageways passing laterally through them, and a shaft extending from the neck, wherein the neck curving along its length from the mounting base to the shaft; and an optional distal stem extending from the proximal body. In a further embodiment, the receiving cavity includes a Morse taper or is cylindrical. In a further embodiment to these embodiments, the cloverleafs with passageways have one or two or three equally sized and spaced passageways between the curve and the mounting base. In a further embodiment to these embodiments, the passageways are configured to receive a suture to secure soft tissue or tuberosities to the proximal body and/or the passageways are configured to receive a suture to secure soft tissue or tuberosities to the proximal body.

In another embodiment, a humeral stem includes a proximal body having a mounting base with a substantially planar surface with notches extending down from a posterior and anterior sides of the mounting base and a Morse taper receiving cavity configured to receive a post of a modular humeral component, a neck extending from the mounting base, the neck having four cloverleafs evenly spaced around the neck, at least three of the cloverleafs (or optionally one or two of the cloverleafs) include two or three equally sized and evenly spaced passageways passing laterally through them, the passageways are configured to receive a suture to secure soft tissue or tuberosities to the proximal body, and a shaft extending from the neck, wherein the neck curving along its length from the mounting surface to the shaft; and a distal stem extending from a distal end of the shaft of the proximal body.

In another embodiment, a humeral stem includes a mounting base having a substantially planar surface with notches extending down from a posterior and anterior sides of the mounting base, and a Morse taper receiving cavity in an axial center of the planar surface, the receiving cavity is configured to receive a post of a modular humeral component; a neck extending from the mounting base, the neck having four cloverleafs evenly spaced around the neck, one to four of the cloverleafs include two, three, or four equally sized and evenly spaced passageways passing laterally through them, the passageways are configured to receive a suture to secure soft tissue or tuberosities to the proximal body; and a shaft extending from the neck, wherein the neck curving along its length from the mounting surface to the shaft. Alternatively to the last two embodiments, the number of passageways per cloverleaf may be one.

In a further embodiment to any of the previous embodiments, the curve between the neck and the shaft is approximately 140 degrees, approximately 145 degrees, or between 135 degrees and 150 degrees with or without the end points as measured by an angle between an axial line perpendicular to the mounting base and an axial line passing through a distal end of the shaft. In a further embodiment to any of the previous embodiments, the distal stem includes a plurality of channels each aligned with a respective area between two cloverleafs of the proximal body.

In a further embodiment to any of the previous embodiments, the proximal body and the distal stem are integrally formed. In a further embodiment to any of the embodiments in the previous paragraphs, the distal end of the shaft includes a connection cavity into which the optional distal stem or an optional cap may be attached and/or otherwise connected to the proximal body. In a further embodiment to the previous embodiment, the proximal body further includes an affixation passageway running from a proximal lateral shoulder to the connection cavity and the affixation passageway and the connection cavity are aligned with each other, the affixation passageway includes a beveled shoulder at its distal end configured to support an inserted screw, and the optional distal stem or the optional cap includes a threaded receiving cavity for receiving the screw inserted through the affixation passageway. In a further embodiment to any of the embodiments of the previous paragraphs, the proximal body further including an affixation passageway proximate the distal end and running from a periphery of the shaft to the connection cavity and perpendicular to the longitudinal direction of the connection cavity, the affixation passageway configured to receiving a means for applying tension on the optional distal stem or optional cap when attached to the proximal body.

In a further embodiment to any of the previous embodiments, the proximal body includes a proximal porous in-growth coating over a majority of a surface of the proximal body; the proximal body has a length of 40 mm and includes a proximal porous in-growth coating over approximately 62.5% of a proximal end and is uncoated over a remaining surface.

In a further embodiment to any of the previous embodiments, curvatures of the lateral sides are different but are complementary to each other and the other sides have curvatures that mirror each other.

In a further embodiment to any of the embodiments with a separate distal stem, the distal stem is selected based on its length to provide a desired overall length for the humeral stem.

### IV. BRIEF DESCRIPTION OF THE DRAWINGS

Any cross-hatching and/or shading present in the figures is not intended to identify or limit the type of material present for the element shown in cross-section or 3-D representation.

FIGS. 1A-1D illustrate an unibody humeral stem according to at least one embodiment of the invention.

FIG. 2 illustrates a proximal body attached to a series of overlayed distal stems according to at least one embodiment of the invention.

FIGS. 3A-3D illustrates an alternative embodiment with two attachable components for the humeral stem according to at least one embodiment of the invention.

FIG. 4A illustrates a partial/phantom cross-section of a proximal body attached to a stem according to at least one embodiment of the invention.

FIG. 4B illustrates a partial cross-section of the proximal body and the stem.

FIG. 5 shows different lengths and sizes of prototype distal stems.

FIG. 6 illustrates a 10 mm proximal body attached to a 10 mm distal stem according to at least one embodiment of the invention.

FIG. 7 illustrates a 12 mm proximal body attached to a 10 mm distal stem according to at least one embodiment of the invention.

FIG. 8 illustrates a 12 mm proximal body attached to a 12 mm distal stem according to at least one embodiment of the invention.

FIG. 9 illustrates a 14 mm proximal body attached to a 12 mm distal stem according to at least one embodiment of the invention.

FIG. 10 illustrates a 14 mm proximal body attached to a 14 mm distal stem according to at least one embodiment of the invention.

FIGS. 11A-11D illustrates an alternative proximal body according to another embodiment of the invention.

FIG. 12 illustrates an example of a cap configured to be inserted into a distal end of the proximal body.

FIG. 13 illustrates an alternative proximal body with an inserted stem according to at least one embodiment of the invention.

### V. DETAILED DESCRIPTION OF THE DRAWINGS

The humeral stem design is congruent with the stemless design for both the ta-TSR and r-TSR. The modularity, in at least one embodiment, also allows for use of the humeral stem in fractures and revision settings. The humeral stem may be used on the humeral side in both ta-TSR and r-TSR procedures as part of a universal prothesis system like those described in in U.S. Pat. No. 10,583,012, issued on Mar. 10, 2020; PCT Application No. PCT/US21/20492, published as WO2021/178418 Al on Sep. 10, 2021 and filed on Mar. 2, 2021; PCT Application No. PCT/US22/26940, published as WO2022/232517 A1 on Nov. 3, 2022 and filed on Apr. 29, 2022; and PCT Application No. PCT/US22/42345, filed on Sep. 1, 2022; and U.S. Provisional Pat. Application No. 63/391,306 (collectively “prior patent applications”). Several of these prior patent applications include examples of different modular humeral components.

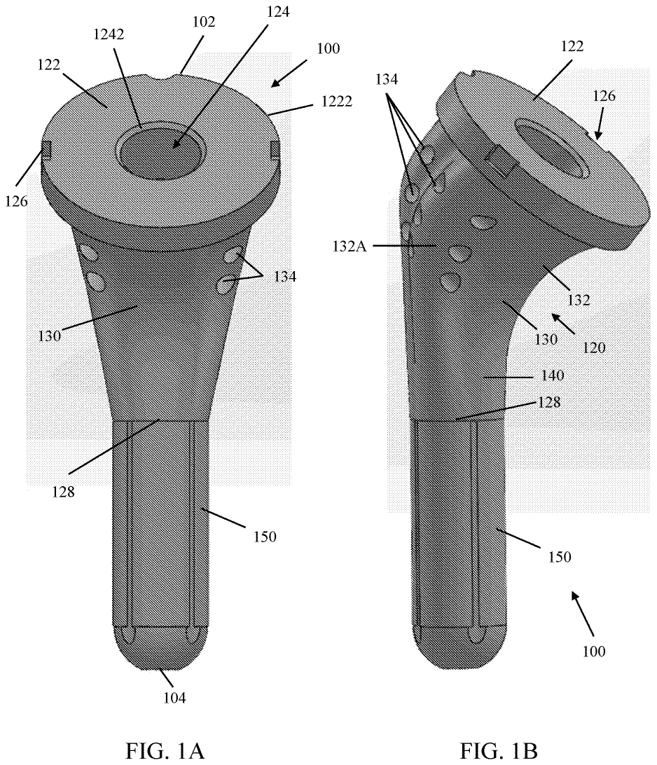

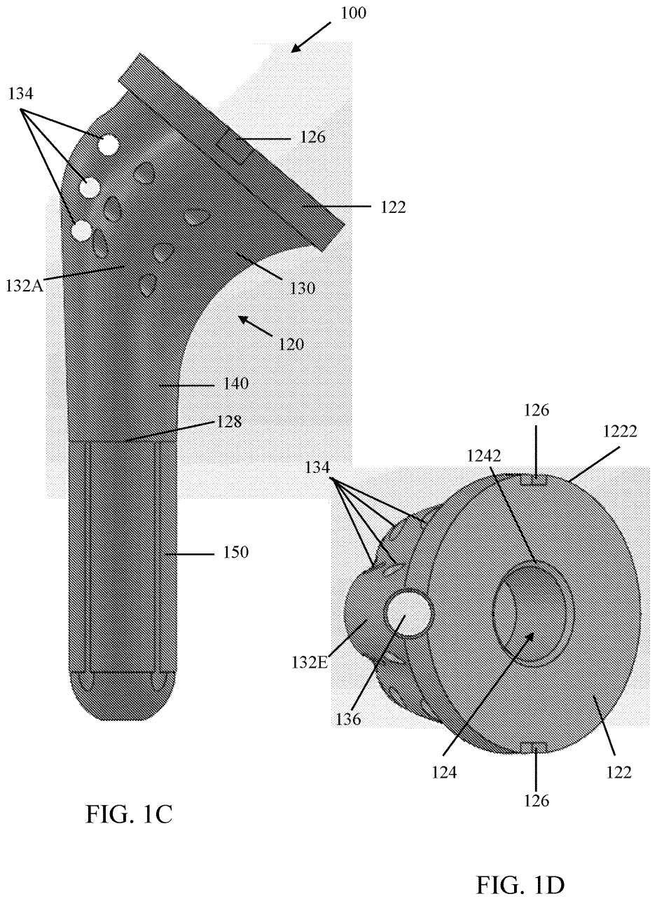

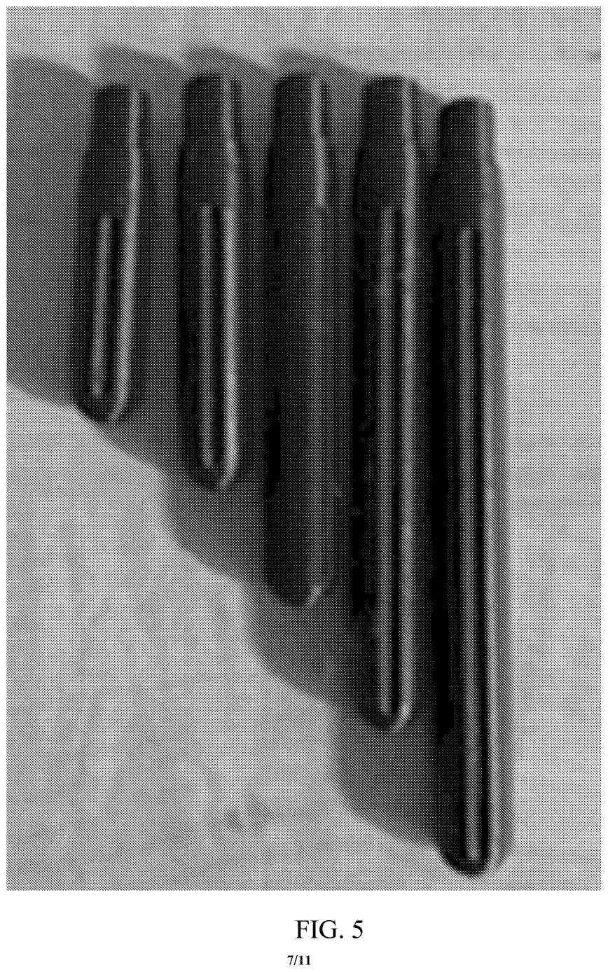

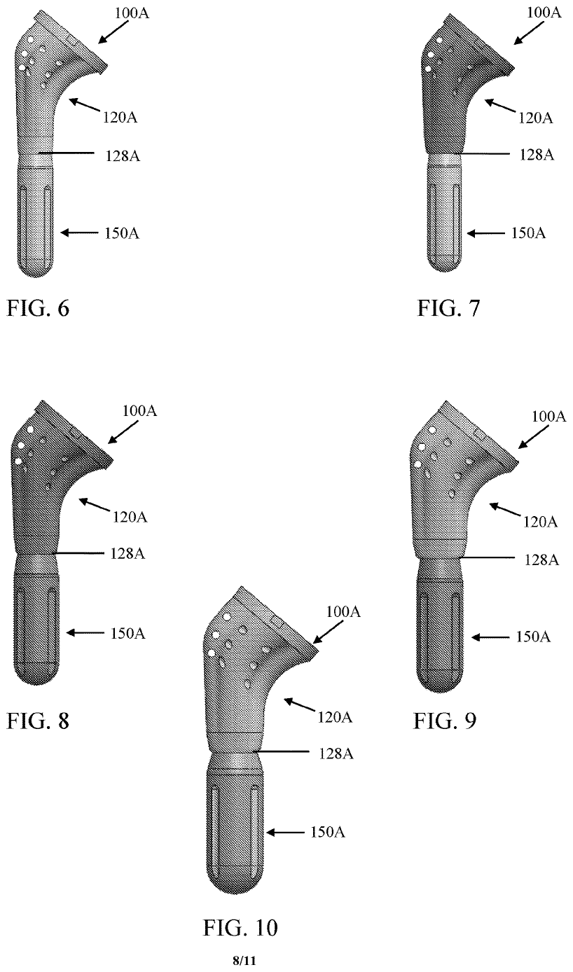

FIGS. 1A-1D illustrate a short stem version of the humeral stem 100 that is an unibody structure. The illustrated humeral stem 100, in at least one embodiment, has an overall length of its proximal body of approximately 40 mm from the proximal lateral shoulder 102 to the distal end 104. The illustrated humeral stem 100 has a distal tip diameter of 10 mm, although other diameter sizes are possible as will be appreciated by a person having ordinary skill in the art based on this disclosure, for example FIGS. 6-10.

The humeral stem 100 has an approximately 140-degree, approximately 145-degree, or between 140-145 degrees or 135-150 degrees (with or without the end points) neck-shaft angle as measured by an angle between a perpendicular line passing through a mounting base 122 and a line passing the axial center of a shaft 140. The use of approximately takes into account differences from how the transition is measured and/or manufacturing tolerances. The humeral stem 100 includes a proximal body 120 and a distal stem 150. The proximal body 120 includes a mounting base 122, a neck 130, and a shaft 140.

A proximal face of the proximal body 120 is the mounting base 122 configured to attach to either the traditional humeral head component or the reverse humeral cup components, for example receiving the modular component's plug into a reverse Morse taper receiving chamber 124 centrally located on the mounting surface 122 of the body 120. The modular humeral components have been previously described in prior patent applications as identified above. In at least one embodiment, the receiving chamber 124 includes a Morse taper. In an alternative embodiment, the receiving chamber 124 is substantially cylindrical. In at least one further embodiment to the other receiving chamber embodiments and as illustrated, the receiving chamber 124 may include a beveled surface 1242 around it opening.

In an alternative embodiment, the illustrated mounting base 122 includes a pair of opposed leverage notches 126 extending down from outer circumferential sides 1222 of the mounting base 122, for example on the anterior and posterior central exterior edge (or side) of the proximal body 120, which in at least one embodiment provides better access to the notches 126 to facilitate separation of the mounted modular humeral component from the proximal body 120 when the humeral body is implanted on the humeral stem 100. The notches 126 are configured to be accessible from the mounting base 122. The notches 126 have sufficient width and depth to receive an instrument in which to pry the attached modular humeral component from the proximal body 120. Although two notches 126 are illustrated, additional notches could be added to the proximal body 120 around the periphery of the mounting base 122.

In at least one embodiment, the mounting base 122 is substantially planar. The plane defined by the mounting base 122 is approximately parallel to the resection plane after implantation. In an alternative embodiment, the plane defined by the mounting base 122 is at an angle to the resection plane after implantation. The mounting base 122 in at least one embodiment will be a sufficient height above the humeral resection plane to allow access to notches 126. In at least one embodiment for each of the illustrated humeral stems, the notches 126 are omitted. Although the mounting base 122 is illustrated as being substantially circular, the mounting base 122 may be elliptical, oval, or other suitable shapes; in such an embodiment, the modular component may be shaped to match.

Examples of mounting base diameters include 25 mm, 27 mm, 29 mm, 32 mm, 33 mm, 36 mm, 37 mm, 39 mm, 41 mm, 42 mm, 43 mm, 46 mm, 49 mm, 52 mm, and 55 mm. Examples of the mounting base thicknesses include 5 mm, 7 mm, 10 mm, 12.5 mm, 14 mm, 15 mm, 17 mm, and 20 mm. In at least one embodiment, the mounting base 122 has an offset center in which there is a neutral position.

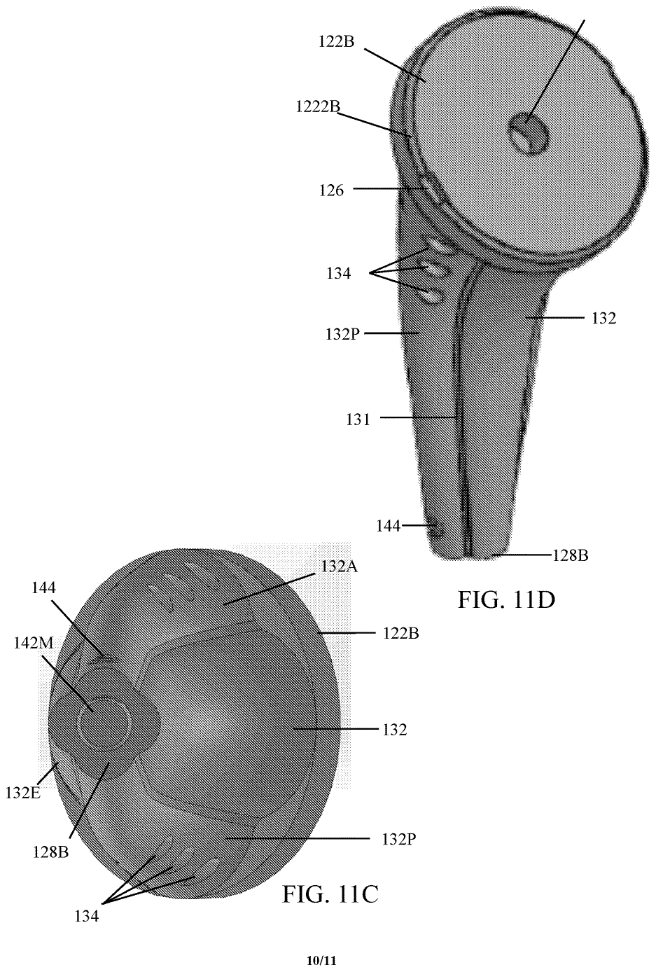

In an alternative embodiment as illustrated in FIGS. 1A-1D, there is a passageway or opening 136 beginning at the proximal shoulder 102 extending in the proximal body 120. In at least one embodiment, the opening 136 may serve as an additional leverage point to remove any attached humeral component and/or a cavity into which a post or other structure of the humeral component may be inserted into and/or secured to after implantation. These features may be in addition or instead of the notches and/or the receiving cavity. The passageway 136 may be omitted from any embodiment with FIGS. 11A-11D and 13 illustrating examples of such an omission.

The illustrated proximal body 120 of the humeral stem 100 includes the neck 130 with a “4-leaf clover” design or similar design with four cloverleafs 132, 132P, 132E, 132A that curves into the shaft 140 to achieve the neck-shaft angle. A cloverleaf is used to describe the structure correlating to the arcuate section in a cross-section take parallel to the bottom of the mounting base 122 and travels from the mounting base 122 to, substantially to, or over the majority of the length to the distal end 128 of the proximal body 120. The proximal body cloverleaf design allows for proximal humeral metaphyseal fill to prevent stress-shielding and bony resorption of the tuberosities and humeral medial calcar. This configuration also provides rotational stability within the proximal humeral canal. Further, there is a gradual proximal to distal taper from the neck 130 to the distal end 128 of the shaft 140 to accommodate both the proximal humeral metaphyseal fill and the transition from metaphyseal to diaphyseal bone.

The illustrated neck 130 includes three cloverleafs 132A, 132E, 132P having suture passageways 134 passing through them parallel to the mounting base 122, although the cloverleafs with passageways may be from zero to the number of cloverleafs present in a particular implementation. In an alternative embodiment, the fourth clover leaf may also have one or more passageways or alternatively the fourth clover leaf is omitted. As illustrated the passageway cloverleafs are on an exterior lateral side 132E, the posterior side 132P, and the anterior side 132A of the neck 130, and in a further embodiment the spacing between the lateral side clover leaf 132E and the posterior/anterior cloverleafs 132P, 132A match. The use of posterior and anterior sides is for reference and reflect the sides if the humeral stem is implanted in the right shoulder as oppose to the left shoulder. As illustrated, the proximal most portions of the clover leaf may have 3 equally sized (e.g., 2 mm) and spaced passageways 134 (e.g., 4 mm separations) through the anterior/posterior and lateral cloverleafs 132A, 132P, 132E to allow for a suture pass through to secure soft tissue or tuberosities, when appropriate, in the settings of fracture fixation (of the tuberosities) or in the revision arthroplasty. In alternative embodiments, the number of suture passageways is 1, 2, or 4 per cloverleaf, and in further embodiments with multiple passageways, the passageways are evenly spaced along the cloverleaf.

Examples of diameters for the distal end 128 of the shaft 140 near the distal tip include any number in the range of 5 mm-10 mm (with or without the end points), 10 mm, 12 mm, 14 mm, 16 mm, 17 mm, any number in the range of 10 mm-14 mm (with or without the end points), any number in the range of 10 mm-18 mm (with or without the end points). Examples of diameters for the distal stem 150 include any number in the range of 5 mm-10 mm (with or without the end points), 10 mm, 12 mm, 14 mm, 16 mm, 17 mm, any number in the range of 10 mm-14 mm (with or without the end points), any number in the range of 10 mm-18 mm (with or without the end points). Different diameter sizes may be present in the proximal body 120 from that of the distal stem 150. FIGS. 1A-1D illustrate the diameter for the proximal body distal end 128 and the distal stem 150 as 10 mm.

In at least one embodiment, the proximal body 120 measures 40 mm in length and includes a proximal porous in-growth coating which covers the proximal 25 mm of the neck where the final 15 mm of the shaft is uncoated.

In at least one embodiment, the shaft 140 and the distal stem 150 are together the shaft 140 with a separate distal stem 150 being omitted.

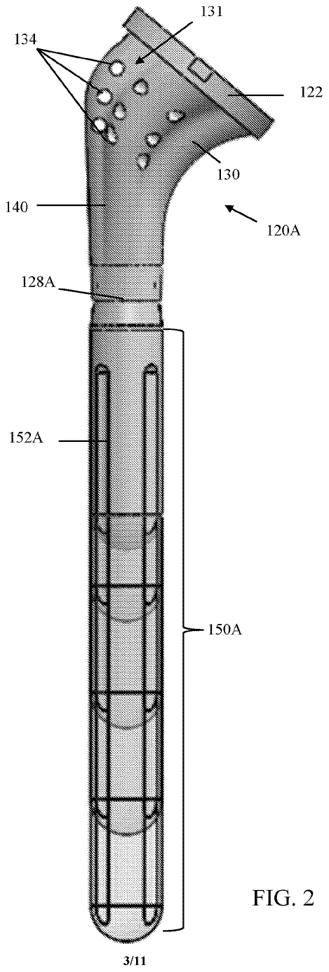

In an alternative embodiment, the proximal body 120A and the distal stem 150A are separate components configured to be connected to each other at the distal end 128A of the proximal body 120A. FIG. 2 illustrates examples of how the distal stem 150A may have a variety of lengths with five length examples being depicted super imposed over each other.

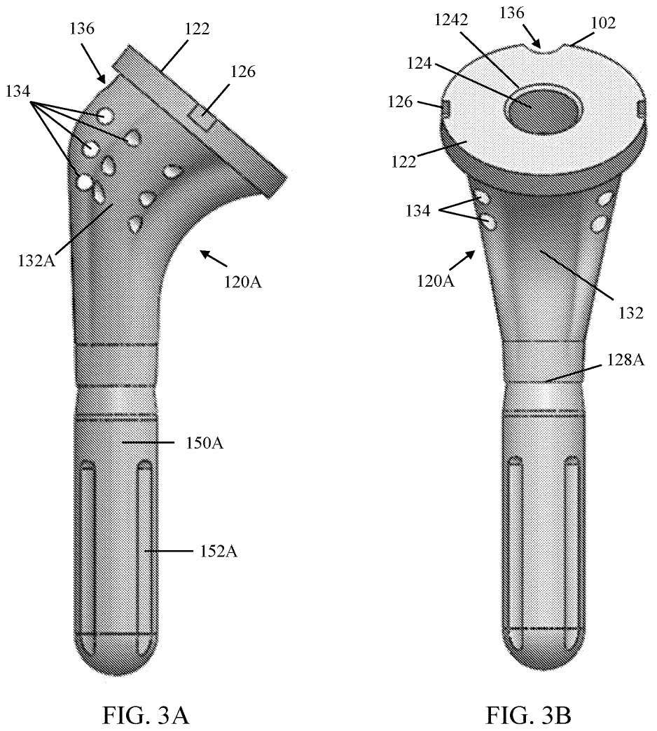

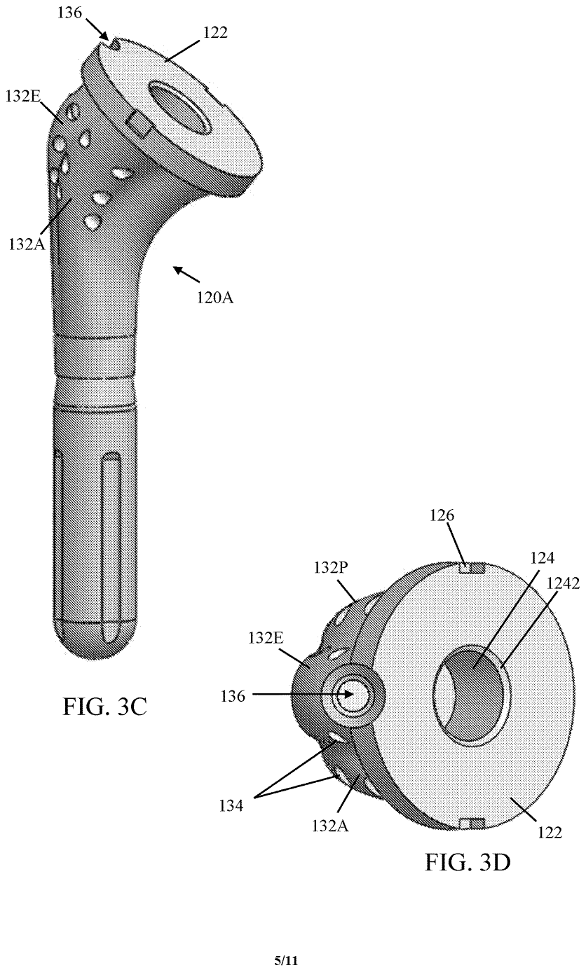

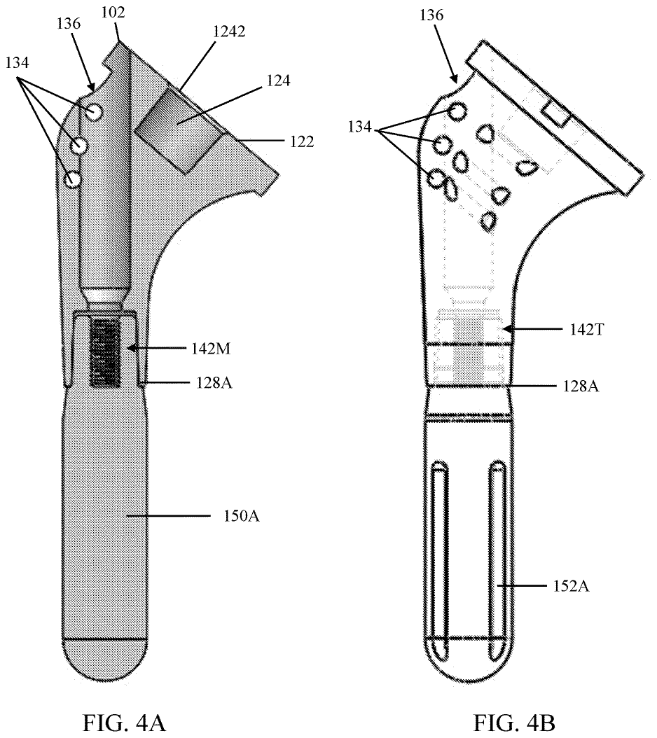

FIGS. 3A-3D illustrate a proximal body 120A attached to a removal distal stem 150A. The illustrated proximal body 120A includes an optional securing passageway 136 for insertion of an affixation screw or other mechanism to secure the distal stem 150A to the proximal body 120A. The illustrated passageway 136 opens at the proximal shoulder 102 and passes behind the suturing passageways 134 of the lateral cloverleaf 132E and is centered with the distal tip 128 of the proximal body 120. In an alternative embodiment, one or more of the suturing passageways 134 of the lateral cloverleaf 132E are omitted or intersect with the securing passageway 136 as illustrated in FIGS. 4A and 4B.

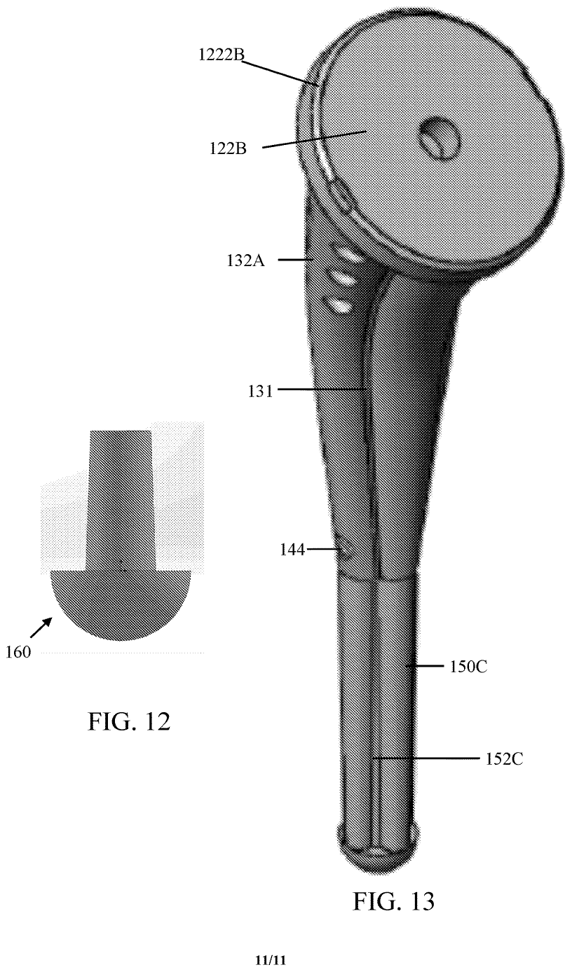

The distal end 128A of the proximal body 120A has a connection cavity 142M, 142T. Examples of the connection cavity include a reverse Morse taper fitting 142M, for example, illustrated in FIG. 4A or a threaded section 142T, for example, illustrated in FIG. 4B. In a further embodiment, a round-head, removable brad/cap 160 (see, e.g., FIG. 12) may be present when no distal stem 150A is attached and where the cap 160 can be removed to allow for modular lengthening of the humeral stem 100A through the introduction of various length distal stems 150A as discussed herein and illustrated, for example, in FIG. 5. In at least one embodiment, the proximal body 120A includes a securing passageway 136 through which an intra-body bolt (or screw) 135 may be inserted for further fixation of the proximal body 120A to the stem(s) 150A or cap 160 as illustrated, for example, in FIGS. 4A and 4B. In a further embodiment, this passageway 136 is omitted and the connection between the proximal body 120B and the distal stem 150 is via a bolt (or screw) inserted through the shaft 140B from the side as illustrated, for example, in FIGS. 11A-11D.

In different alternative embodiments, the proximal body 120A can be assembled with various length stems 150A to optimize centralization and stability as illustrated, for example, in FIGS. 2 and 5-10. FIG. 5 shows five examples of prototype distal stems. Stem length constructions vary according to the setting of primary arthroplasty, fracture, or revision arthroplasty. For example, to construct a short humeral stem, a 35 mm stem can be utilized and attached to the proximal body 120A, making the total short stem length 75 mm. The current guidelines have the insertion depth on the humeral side as being 85 mm. The standard humeral stem would use a 45 mm stem to the proximal body for a total length of 85 mm. Additional examples of stem lengths that may increase in 15 mm increments for stem lengths of: 45 mm, 60 mm, 75 mm, 90 mm, and 105 mm. The attachable stem can be impacted into the distal portion of the proximal body utilizing the male-female Morse taper like that illustrated, for example, in FIG. 4A. In a further embodiment, a torque limiting screw passed through the proximal body 120A into the distal stem 150A can secondarily secure the secure the distal stem 150A to the proximal body 120A as illustrated, for example, in FIGS. 4A and 4B. The securing passageway 138 is aligned with the connection cavity 142M, 142T of the shaft 140.

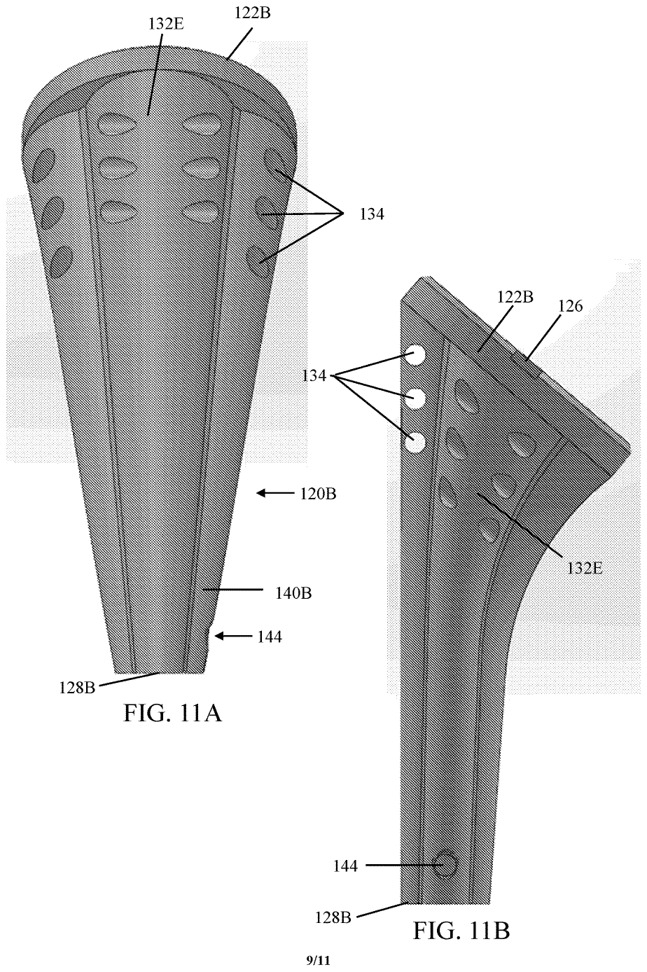

An advantage to the proximal body 120A and the distal stem 150A being two separate components is that it allows the surgeon to have different diameters between these two components as illustrated, for example, in FIGS. 6-10 where the proximal body diameter is at the distal end 128A and the distal stem diameter is prior to the rounded end illustrated in these figures. FIG. 6 illustrates a 10 mm proximal body attached to a 10 mm distal stem. FIG. 7 illustrates a 12 mm proximal body attached to a 10 mm distal stem. FIG. 8 illustrates a 12 mm proximal body attached to a 12 mm distal stem. FIG. 9 illustrates a 14 mm proximal body attached to a 12 mm distal stem. FIG. 10 illustrates a 14 mm proximal body attached to a 14 distal stem.

FIGS. 11A-11D illustrate an alternative embodiment of the proximal body 120B that includes a lateral securing channel 144 near the distal end 128 of the neck 140B for insertion of an attachment screw or bolt or other similar mechanism for securing the distal stem to the proximal body 120B instead of the securing passageway 136 illustrated in FIGS. 4A and 4B. FIG. 11B. 11D. and 13 illustrate an alternative embodiment where the mounting base 122B includes a beveled outer periphery edge 1222B. These figures otherwise share similar features to the previously described embodiments, for example as reflected by the reference numbers.

FIG. 12 illustrates an example of a distal cap 160 that may be used to seal the bottom of the proximal body 120A, 120B or, alternatively the bottom of a distal stem.

FIG. 13 illustrates a further alternative embodiment showing a different configuration of the distal stem 150C in terms of the horizontal cross-section from the previously illustrated distal stems 150A. Various figures illustrate the presence of channels 152, 152A, 152C in the distal stem 150, 150A, 150C. The channels 152, 152A, 152C improve rotational stability after the humeral stem 100 is implanted, but also provide for an easier gripping surface. In at least one embodiment, the channels 152, 152A, 152C are aligned with the areas 131 between the cloverleafs 132 to provide a smoother transition from the proximal body 120 to the stem 150.

Although particular materials have been identified for particular components and structural elements, one of ordinary skill in the art will appreciate that other materials may be substituted without departing from the scope of the invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

As used above “substantially,” “generally,” “approximately,” and other words of degree are relative modifiers intended to indicate permissible variation from the characteristic so modified particularly when relating to manufacturing and production tolerances. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

Although one level of multiple dependencies is present in the claims attached to this disclosure, it should be understood that none conflicting claim recitation of dependent claims may be combined, for example, in the manner of the priority applications.

1. A humeral stem comprising:
a proximal body having
a mounting base with a substantially planar mounting surface, the mounting base configured to attach to a modular humeral component,
a neck extending from said mounting base, said neck having three cloverleafs evenly spaced on three sides or four cloverleafs evenly spaced around said neck such that a cross-section resembles a circle with arcuate areas around the circle's perimeter, at least three of the cloverleafs include one or more passageways passing laterally through them, and
a shaft extending from said neck, wherein said neck curving along its length from the mounting base to said shaft.

2. The humeral stem according to claim 1, wherein the curve between said neck and said shaft is approximately 140 degrees, approximately 145 degrees, or between 135 degrees and 150 degrees with or without the end points as measured by an angle between an axial line perpendicular to said mounting base and an axial line passing through a distal end of said shaft.

3. (canceled)

4. The humeral stem according to claim 1, wherein the cloverleafs with passageways have three equally sized and spaced passageways between the curve and said mounting base.

5. (canceled)

6. The humeral stem according to claim 1, wherein the passageways are configured to receive a suture to secure soft tissue or tuberosities to the proximal body.

7. The humeral stem according to claim 1, wherein said distal stem includes a plurality of channels each aligned with a respective area between two cloverleafs of said proximal body.

8. The humeral stem according to claim 1, wherein said proximal body and said distal stem are integrally formed.

9. The humeral stem according to claim 1, wherein said distal end of said shaft includes a connection cavity into which said a optional distal stem or a distal cap may be attached and/or otherwise connected to said proximal body.

10. The humeral stem according to claim 9, wherein said proximal body further includes an affixation passageway running from a proximal lateral shoulder to the connection cavity and said affixation passageway and the connection cavity are aligned with each other, said affixation passageway includes a beveled shoulder at its distal end configured to support an inserted screw, and
said distal stem or said distal cap includes a threaded receiving cavity for receiving the screw inserted through said affixation passageway.

11. The humeral stem according to claim 9, where said proximal body further including an affixation passageway proximate said distal end and running from a periphery of said shaft to the connection cavity and perpendicular to the longitudinal direction of the connection cavity, said affixation passageway configured to receiving a means for applying tension on said distal stem or said distal cap when attached to said proximal body.

12. The humeral stem according to claim 1, wherein
said proximal body includes a proximal porous in-growth coating over a majority of a surface of said proximal body, or
said proximal body has a length of 40 mm and includes a proximal porous in-growth coating over approximately 62.5% of a proximal end and is uncoated over a remaining surface.

13. (canceled)

14. The humeral stem according to any one of claim 1, wherein curvatures of the lateral sides are different but are complementary to each other and the other sides have curvatures that mirror each other.

15. A humeral stem comprising:
a proximal body having
a mounting base with a substantially planar mounting surface with notches extending down from a posterior and anterior sides of said mounting base and a Morse taper receiving cavity configured to receive a post of a modular humeral component, the mounting base having no structure above the mounting surface,
a neck extending from said mounting base, said neck having four cloverleafs evenly spaced around said neck, at least three of the cloverleafs include three equally sized and evenly spaced passageways passing laterally through them, the passageways are configured to receive a suture to secure soft tissue or tuberosities to the proximal body, and
a shaft extending from said neck, wherein said neck curving along its length from said mounting surface to said shaft; and
a distal stem or a distal cap extending from a distal end of said shaft of said proximal body.

16. The humeral stem according to claim 15, wherein the curve between said neck and said shaft is approximately 140 degrees, approximately 145 degrees, or between 135 degrees and 150 degrees with or without the end points as measured by an angle between an axial line perpendicular to said mounting base and an axial line passing through said distal end of said shaft.

17. The humeral stem according to claim 15, wherein said distal stem includes a plurality of channels each aligned with a respective area between two cloverleafs of said proximal body.

18. (canceled)

19. The humeral stem according to claim 15, wherein said distal end of said shaft includes a connection cavity into which said distal stem or said distal cap may be attached and/or otherwise connected.

20. The humeral stem according to claim 19, wherein said proximal body further includes an affixation passageway running from a proximal lateral shoulder to the connection cavity and said affixation passageway and the connection cavity are aligned with each other, said affixation passageway includes a beveled shoulder at its distal end configured to support an inserted screw, and
said distal stem includes a threaded receiving cavity for receiving the screw inserted through said affixation passageway.

21. The humeral stem according to claim 19, wherein said proximal body further including an affixation passageway proximate the distal tip and running from the periphery of the shaft to the connection cavity and perpendicular to the longitudinal direction of the connection cavity, said affixation passageway configured to receiving a means for applying tension on said distal stem when attached to said proximal body.

22. The humeral stem according to claim 19, wherein said distal stem is selected based on its length to provide a desired overall length for said humeral stem.

23. The humeral stem according to claim 15, wherein
said proximal body includes a proximal porous in-growth coating over a majority of a surface of the proximal body, or
said proximal body has a length of 40 mm and includes a proximal porous in-growth coating over approximately 62.5% of a proximal end and is uncoated over the remaining surface.

24. (canceled)

25. (canceled)

26. A humeral stem comprising:
a mounting base having
a substantially planar mounting surface with notches extending down from a posterior and anterior sides of said mounting base, the mounting base having no structure above the mounting surface, and
a Morse taper receiving cavity in an axial center of said planar surface, the receiving cavity is configured to receive a post of a modular humeral component;
a neck extending from said mounting base, said neck having four cloverleafs evenly spaced around said neck, at least one of the cloverleafs include three equally sized and evenly spaced passageways passing laterally through them, the passageways are configured to receive a suture to secure soft tissue or tuberosities to the proximal body; and
a shaft extending from said neck, wherein said neck curving along its length from the mounting surface to said shaft.

27. (canceled)
